(19) United States
(12) Patent Application Publication
(10) Pub. No.: US 2019/0130332 A1
(43) Pub. Date: May 2, 2019

(54) CLINICAL TELEMETRY PATIENT MONITORING BATTERY MANAGEMENT SYSTEM AND METHOD

(71) Applicant: GENERAL ELECTRIC COMPANY, Schenectady, NY (US)

(72) Inventor: Brian Janssen, Brookfield, WI (US)

(21) Appl. No.: 15/798,966

(22) Filed: Oct. 31, 2017

(51) Int. Cl. G06Q 10/06; A61B 5/00; G01R 31/36

(52) U.S. Cl. CPC G06Q 10/06315; A61B 5/0002; A61B 2560/0214; G01R 31/3606; G01R 31/3679; G01R 31/3651

(57) ABSTRACT

Systems and methods for managing a fleet of batteries used in a clinical patient monitoring system are disclosed. The method includes receiving statuses of batteries in the fleet from transmitters/transceivers in which the batteries are used. Each transmitter/transceiver is associated with a corresponding patient for monitoring the health of the patient. The method also includes predicting a remaining life at a given point of time for each of the batteries based on the received status and managing battery replacement based on the remaining life. The method further comprises conducting statistical analyses on operational metrics of the fleet of batteries and managing the operation of the fleet of batteries based on the statistical analyses.

### BACKGROUND

This disclosure relates generally to battery management systems and methods, and more specifically to systems and methods for managing a fleet of batteries used in a telemetry physiological patient monitoring system.

Battery powered telemetry patient monitoring devices are widely used in care delivery in areas such as patient monitoring, fetal monitoring, and rehabilitation services. Physiological conditions of patients or fetuses, such as ECG, blood pressure, temperature, heart rate, oxygen saturation, etc., can be transmitted by telemetry devices to a remote processing system. It can be time consuming, interruption prone, and costly to constantly manage a large fleet of batteries where a plurality of telemetry patient monitoring devices are operated and maintained in an hospital or clinic environment. For example, a low battery status may trigger technical alarms requiring immediate battery change, which, on top of patient physiological alarms and other system alarms, may increase alarm fatigue on medical professionals. In addition, care delivery processes as well as patients may be disrupted when batteries are changed at inconvenient times such as in the middle of the night when patients are trying to get critical sleep. Furthermore, the total cost of owning and operating the fleet, including battery acquisition costs, staff costs, and costs related to alarm fatigue, can be very high. It is generally desirable to improve and optimize the battery management process to reduce technical alarms, enhance patient satisfaction, and reduce the cost of operating and supporting the clinical telemetry patient monitoring system.

### SUMMARY

In one embodiment, the present disclosure provides a method for managing a fleet of batteries used in a clinical telemetry patient monitoring system. The method comprises receiving statuses of batteries in the fleet from transmitters and/or transceivers in which the batteries are used. Each transmitter or transceiver is associated with a corresponding patient for monitoring physiological conditions of the patient. The method also comprises predicting a remaining life at a given point of time for each of the batteries based on the received statuses and managing battery replacement based on the remaining life.

In another embodiment, the present disclosure provides a processing system for managing a fleet of batteries used in a clinical telemetry patient monitoring system. The processing system comprises a fleet status compiler configured to compile statuses of batteries in the fleet collected from transmitters and/or transceivers in which the batteries are used. Each transmitter or transceiver is associated with a corresponding patient for monitoring physiological conditions of the patient. The processing system also comprises an analytics engine configured to predict a remaining life at a given point of time for each of the batteries based on the statuses of batteries and manage battery replacement based on the remaining life.

In yet another embodiment, the present disclosure provides a non-transitory computer readable medium comprising instructions which, when executed by a processing system, cause the processing system to perform operations. The operations comprise receiving statuses of batteries in a fleet from transmitters and/or transceivers in which the batteries are used. Each transmitter or transceiver is associated with a corresponding patient for monitoring health of the patient. The operations also comprise predicting a remaining life at a given point of time for each of the batteries based on the received status and managing battery replacement based on the remaining life.

### BRIEF DESCRIPTION OF THE DRAWINGS

Various aspects of this disclosure may be better understood upon reading the following detailed description and upon reference to the drawings in which:

FIG. 1 is a block diagram of a clinical telemetry patient monitoring system, in accordance with an exemplary embodiment;

FIG. 2 is a block diagram of a battery management system that can be used for the clinical telemetry patient monitoring system of FIG. 1, in accordance with an exemplary embodiment;

FIG. 3 is a fleet status summary, in accordance with an exemplary embodiment;

FIG. 4A is a graph which shows fitting battery level readings with a fidelity in ⅛ increments to a battery level decay line, in accordance with an exemplary embodiment;

FIG. 4B is a graph which shows fitting battery level readings with a fidelity in 1/100 increments to a battery level decay line, in accordance with an exemplary embodiment;

FIG. 5 is a graph which shows battery level decay lines with and without SpO2 monitoring, in accordance with an exemplary embodiment;

FIG. 6 is a fleet report generated by the battery management system of FIG. 2, in accordance with an exemplary embodiment;

FIG. 7A is a flow chart of a method for determining whether to replace a battery in a batched replacement event before an alarm is generated, in accordance with an exemplary embodiment;

FIG. 7B is a flow chart of a method for determining whether to replace a battery in a batched replacement event before an alarm is generated, in accordance with another exemplary embodiment;

FIG. 7C is a flow chart of a method for determining whether to replace a battery in a single replacement event before an alarm is generated, in accordance with an exemplary embodiment;

FIG. 7D is a flow chart of a method for determining whether to reuse a battery when an associated patient is discharged, in accordance with an exemplary embodiment;

FIG. 8 is a printed order of battery change request generated by the battery management system of FIG. 2, in accordance with an exemplary embodiment;

FIG. 9 is a chart showing statistics on battery usage and costs generated by the battery management system of FIG. 2, in accordance with an exemplary embodiment;

FIG. 10 is a chart showing statistics on battery change request compliance generated by the battery management system of FIG. 2, in accordance with an exemplary embodiment;

FIG. 11A is a chart showing statistics on battery replacement breakdown by the time of day before the replacement process was modified by the battery management system FIG. 2, in accordance with an exemplary embodiment;

FIG. 11B is a chart showing statistics on battery replacement breakdown by the time of day after the replacement process was modified by the battery management system FIG. 2, in accordance with an exemplary embodiment;

FIG. 12 is a chart showing statistics on batteries changed at various remaining battery life in terms of battery level, in accordance with an exemplary embodiment;

FIG. 13 is a chart showing statistics on batteries changed at various remaining battery life in terms of percentage of total battery life, in accordance with an exemplary embodiment; and

FIG. 14 is a flow chart of a method for managing a fleet of batteries used in the clinical telemetry patient monitoring system of FIG. 1, in accordance with an exemplary embodiment.

The drawings illustrate specific aspects of the described components, systems and methods for managing a battery fleet for a clinical telemetry patient monitoring system. Together with the following description, the drawings demonstrate and explain the principles of the structures, methods, and principles described herein. In the drawings, the size of components may be exaggerated or otherwise modified for clarity. Well-known structures, materials, or operations are not shown or described in detail to avoid obscuring aspects of the described components, systems, and methods.

### DETAILED DESCRIPTION

One or more specific embodiments of the present disclosure are described below in order to provide a thorough understanding. These described embodiments are only examples of the systems and methods for managing a battery fleet for a clinical telemetry patient monitoring system. The skilled artisan will understand that specific details described in the embodiments can be modified when being placed into practice without deviating the spirit of the present disclosure.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

Referring to the figures generally, the present disclosure is to provide systems and methods for managing a battery fleet for a clinical telemetry patient monitoring system. For the simplicity of expression, the term “clinical telemetry patient monitoring system” will be used hereafter in the disclosure, which is intended to address the telemetry-based systems used in patient monitoring, fetal monitoring, rehabilitation services, and the like. Batteries in the fleet are used on transmitters or transceiver devices that monitor health or physiological condition of patients. An exemplary battery management system can be run on various processing systems in the telemetry system, such as a telemetry server, a central station, a gateway, or any other appropriate processing system(s). The battery management system receives the statuses of batteries in the fleet from the transmitters/transceivers on which the batteries are used. The battery management system predicts a remaining life at a given point of time for each of the batteries based on the received statuses. Based on the predicted remaining life, the battery management system manages battery replacement to, for example, avoid battery alarms during the night while patients may be sleeping. Furthermore, the battery management system conducts statistical analyses on operational metrics (e.g., cost of ownership, disruption to patients, cost to change, percentage of battery or set of batteries used at time of change, time of day percentage of batteries changed) of the fleet of batteries. Based on the statistical analyses, the battery management system implements policies to optimize at least some of the metrics.

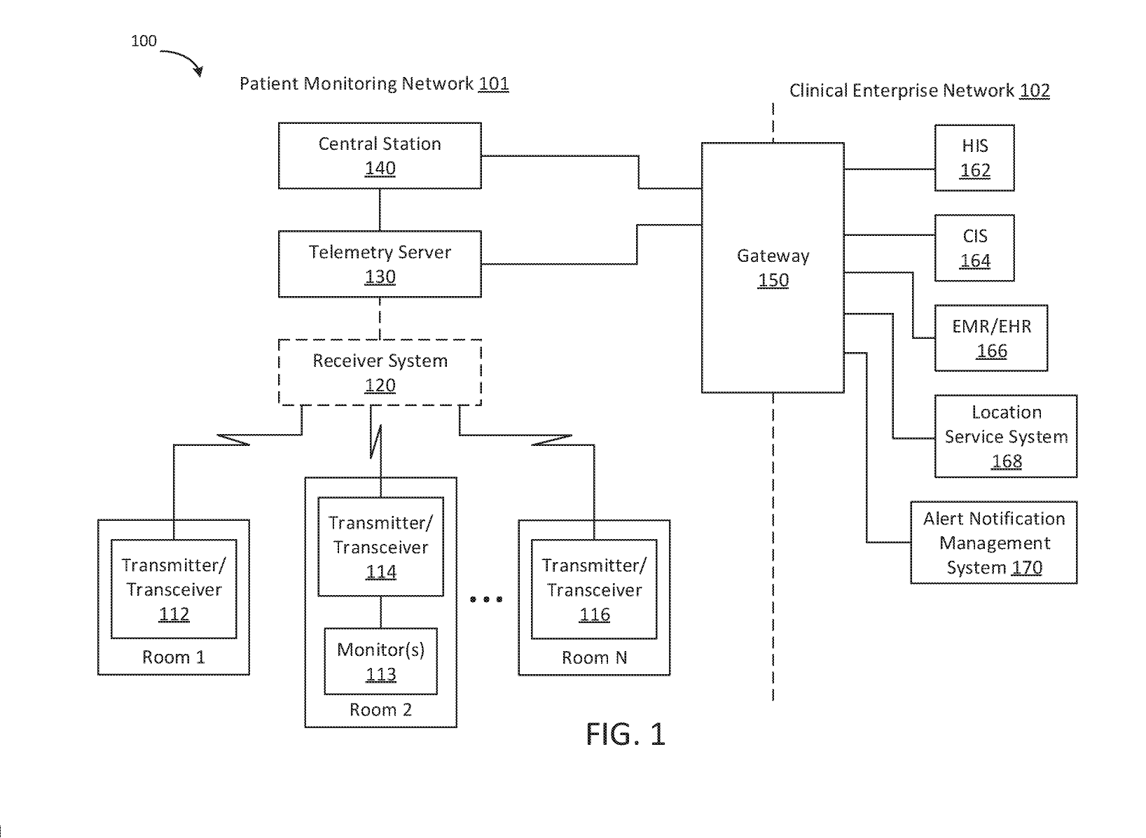

Now referring to FIG. 1, a block diagram of a clinical telemetry patient monitoring system 100 is shown, in accordance with an exemplary embodiment. As illustrated in FIG. 1, in some embodiments, the telemetry system 100 includes a patient monitoring network 101 and a clinical enterprise network 102 connected via a gateway 150. The patient monitoring network 101 is a networking system for patient monitoring devices connected to the network to transport life-critical data. The clinical enterprise network 102 is an IT infrastructure that facilitates the operation and management of a clinical enterprise. The gateway 150 is the interface between the patient monitoring network 101 and the clinical enterprise network 102. In some embodiments, the gateway 150 collects data acquired by patient monitoring devices from the patient monitoring network 101, translates the data by using, for example, HL7 protocol, and transfers the translated data to the clinical enterprise network 102. On the other hand, the gateway 150 collects patient information (e.g., admission-discharge-transfer data, demographic data) from the clinical enterprise network 102, translates the patient information, and transfers the translated patient information to the patient monitoring network 101.

It should be understood the architecture of the clinical telemetry patient monitoring system 100 as shown in FIG. 1 is for illustration and not for limitation. A clinical telemetry patient monitoring system may have a different architecture, for example, the patient monitoring network can be part of the clinical enterprise network rather than a separate network and the gateway 150 can be omitted. It should also be understood that the clinical telemetry patient monitoring system may include more, fewer, and/or different components than those shown in FIG. 1.

As illustrated in FIG. 1, in some embodiments, the patient monitoring network 101 includes a plurality of transmitters/transceivers 112, 114, and 116 disposed in various rooms of the clinic, monitor(s) 113, receiver system 120, telemetry server 130, and central station 140. For the simplicity of expression, the term “transmitter” will be used thereafter in the disclosure, which is intended to include both “transmitter” and “transceiver.” Transmitters 112, 114, or 116 continuously monitor physiological characteristics of patients. In some embodiments, monitor(s) 113 is used to display the monitored physiological characteristics of the patient at bedside. Transmitters 112, 114, and 116 transmit patient physiological data to the receiver system 120 through wireless communication links (e.g., antennas, access points). The telemetry server 130 receives the data from the receiver system 120 and sends the data to the central station 140 for processing/viewing via, for example, a dedicated network interface. In some embodiments, the receiver system 120 can be omitted from the patient monitoring network 101. Transmitters 112, 114, and 116 transmit patient physiological data to the telemetry server 130 and/or the central station 140 through the access point (AP) based solution.

In some embodiments, the transmitters 112, 114, and 116 support body-worn (e.g., handheld or wearable) devices that are attached to patients at various rooms (e.g., Room 1, Room 2, . . . Room N) of the clinic and sense physiological characteristics from the patient. For example, the device and associated accessories may include ECG electrodes for sensing ECG, connecting ECG cables if not connected wirelessly, a wired or wireless connected blood pressure cuff and acquisition device for sensing non-invasive blood pressure (NIBP), and/or a wired or wireless pulse oximetry sensor for sensing peripheral oxygen saturation (SpO2). Other supported telemetry applications such as fetal monitoring may use these, a subset, or additional accessories to monitor a patient and/or fetus. In some embodiments, one or more monitors 113 are connected to a transmitter (e.g., transmitter 114) in the same room for displaying the monitored physiological characteristics of the patient at bedside. Transmitters 112, 114, and 116 transmit patient physiological data acquired by the sensing devices to the receiver system 120 or telemetry server 130 or other processing system via antennas, access points, or similar infrastructure. The data transmitted may include the raw physiological data, filtered physiological data, and/or processed data indicating information about the respective physiological parameter measured from the patient. In an example embodiment, each of the transmitters 112, 114, and 116 has a transmitter ID (e.g., ttx number) programmed into the transmitters. The ttx number of the transmitter can be attached to the data transmitted from the transmitter.

Transmitters 112, 114, and 116 are powered by batteries. For example, a transmitter can run on two AA batteries, one 9-volt battery, or other battery sizes and numbers of batteries. The battery life can be tens to more than a hundred hours, depending on the power and operation of the particular transmitter. For example, different brands/models of transmitters may have different nominal power usage requirements. As another example, for a transmitter capable of monitoring ECG, NIBP, and SpO2, the battery life is likely shorter when the SpO2 monitoring is turned on than when the SpO2 monitoring is turned off. In some embodiments, the transmitter has a “change battery” indicator (e.g., an LED). When the battery power is running low, the change battery LED flashes. Transmitters 112, 114, and 116 may continuously measure their battery statuses (e.g., battery level) and transmit the readings to the receiver system 120 or the telemetry server 130 or another processing system via wireless communication links.

In some embodiment, the sensing devices and associated accessories do not use separate batteries than the batteries in transmitters 112, 114, and 116. In some embodiments, some or all of the sensing devices and associated accessories may be powered by separate batteries. The batteries in the sensing devices and accessories also need to be managed. In one case, statuses of the batteries used in the sensing devices and accessories are transmitted by the transmitters 112, 114, and 116 to the receiver system 120 or the telemetry server 130 or another processing system. In another case, the sensing devices and accessories have wireless communication interfaces and can transmit the battery statuses by themselves. Systems and methods discussed in the present disclosure apply to all these situations.

The receiver system 120 or the telemetry server or another processing system receives data from transmitters 112, 114, and 116 via wireless communication links that may employ various wireless communication protocols used with antennas, access point, or similar infrastructure, such as Bluetooth, Bluetooth Low Energy (BLE), ZigBee, Wi-Fi, infrared, ultrasound, and so on.

The telemetry server 130 receives data from the receiver system 120 via, for example, a dedicated network interface (e.g., dedicated Ethernet) or directly from the transmitters through wireless links. In some embodiments, the telemetry server 130 includes a computing system that runs telemetry host application software. The telemetry host application software processes the data received from the receiver system 120 and makes physiological parameters (and waveforms if applicable) of patients available for displaying and further processing at the central station 140. In some embodiments, the telemetry server 130 is integrated into the central station 140.

At the central station 140, monitored physiological characteristics for multiple patients may be displayed simultaneously. In addition, the central station 140 may generate various types of alarms based on data from transmitters 112, 114, and 116. For example, patient physiological alarms are triggered if alarm limits for the respective physiological parameter data are exceeded, or certain data patterns are detected. Technical alarms can be generated based on function of the transmitters. Examples of technical alarms include low battery alerts, transmitter off alerts (e.g., the transmitter is not properly attached to the patient), transmitter out of range or off the network, transmitter malfunction alerts (e.g., transmitter is not functioning properly), data transmission malfunction alert (e.g., there is a problem with one or more communication links), or alerts on other technical problems. In some embodiments, alarms include audible noises and/or flashing lights. In some embodiments, the central station 140 or telemetry server 130 generates alarm notifications for sending to mobile devices (e.g., pagers, PDAs, mobile phones) of caregivers via, for example, an alert or alarm notification system (e.g., ASCOM alarm notification system).

The clinical enterprise network 102 may include one or more of a hospital information system (HIS) 162, clinical information system (CIS) 164, electronic medical record system (EMR)/electronic health record system (EHR) 166, location service system 168, and alert/alarm notification management system 170. The HIS 162, CIS 164, and/or EMR/HER 166 manage patient registration information, admission-discharge-transfer information, billing information, scheduling information, prescription information, and so on. The location service system 168 obtains and manages patient/device location information. The alert/alarm notification management system 170 is used as a communication and notification system when messages or alerts based on a determination by the battery management system need to be communicated to a medical, technical, support, etc. person. As discussed above, the clinical enterprise network 102 can exchange information with the patient monitoring network 101 via the gateway 150.

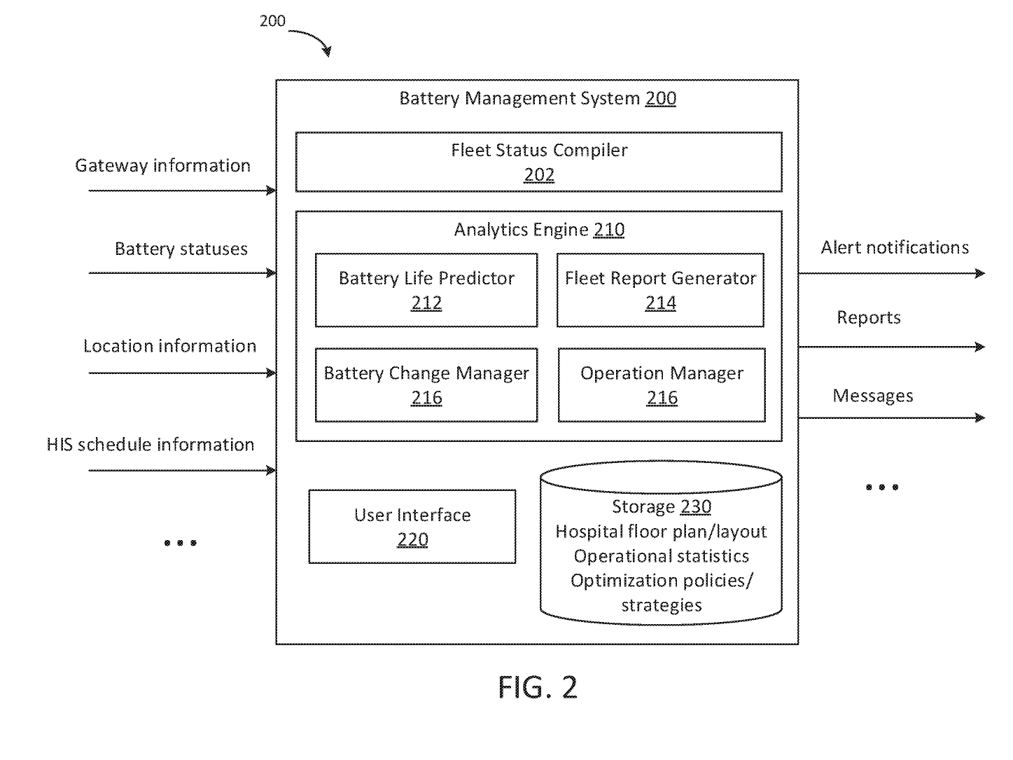

Referring now to FIG. 2, a block diagram of a battery management system 200 is shown, in accordance with an exemplary embodiment. The battery management system 200 can be used for the clinical telemetry patient monitoring system 100 of FIG. 1, managing batteries used in transmitters 112, 114, and 116 (and batteries used in sensing devices and accessories if applicable). In some embodiments, the battery management system 200 may be run on any of the telemetry server 130, central station 140, gateway 150, HIS 162, CIS 164, EMR/EHR 166, or a separate processing system (not shown in FIG. 1) in the patient monitoring network 101 or the clinical enterprise network 102, or interfacing the networks 101 and 102. In some embodiments, the battery management system 200 may be implemented on more than one of the above processing systems, i.e., a portion of the battery management system 200 is run on one processing system, and another portion of the battery management system 200 is run on another processing system.

The battery management system 200 can be implemented as software (e.g., programmed instructions), hardware (e.g., application-specific integrated circuit, field programmable gate array), firmware (e.g., microprocessors), or any combination thereof. For example, an embodiment may employ various integrated circuit components, e.g., memory elements, logic elements, or the like, which may carry out a variety of functions under the control of one or more processors or other control devices.

As illustrated in FIG. 2, in some embodiments, the battery management system 200 includes a fleet status compiler 202, analytics engine 210, user interface 220, and optionally storage 230. In operation, the fleet status compiler 202 compiles information on fleet status, the analytics engine 210 conducts analytical analyses on the fleet status and operational metrics of the fleet, manages battery replacement, and manages operation of the fleet. The user interface 220 allows a user (e.g., a medical professional or staff) to interact with the battery management system 200, such as receiving input from the user and presenting information to the user. The user interface 220 can include any combination of software (e.g., graphical user interface) and hardware (e.g., screen, speaker, keyboard, mouse, etc.). It should be understood a battery management system can include fewer, more, or different components than those shown in FIG. 2 for performing the functions described herein.

The fleet status compiler 202 is configured to compile information on statuses of batteries in the fleet collected from transmitters 112, 114, and 116. In some embodiments, the telemetry server 130 maintains a log file where readings of battery level for transmitters 112, 114, and 116 at various points of time are recorded. In some embodiments, a transmitter sends its current battery level to the telemetry server 130 when changing to a different battery level, changing to specified voltage levels, or at a predefined or adjustable interval (e.g., 30 minutes, 60 minutes, etc.). In other embodiments, the telemetry server 130 queries the transmitters for battery level readings at a predefined or adjustable interval, and the transmitters send the readings in response to the query. The fleet status compiler 202 may receive the fleet status information from the log file in one non-limiting example. In some embodiments, the battery statuses information may be sent wirlessly to the battery management system 200 directly by the transmitters or from the telemetry server 130.

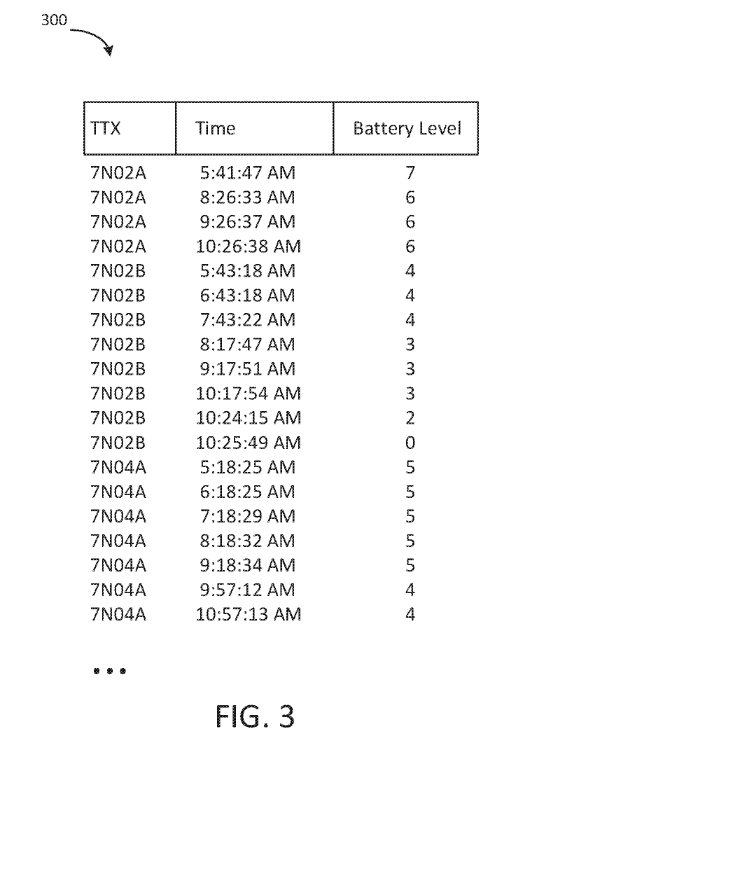

FIG. 3 shows a fleet status summary 300 complied by the fleet status compiler 202, in accordance with an exemplary embodiment. The fleet status summary 300 includes readings of battery level associated with three transmitters (i.e., ttx 7N02A, 7N02B, and 7N04A) at various points of time. The battery level fidelity may vary in different embodiments. For the example shown in FIG. 3, the battery level fidelity is in ⅛ increments, i.e., a battery level reading can be one of 0, 1, 2, 3, 4, 5, 6, or 7. In other embodiments, the battery level fidelity can be, for example, 1/16, 1/32, 1/100, 1/1000, and so on.

Referring back to FIG. 2, the analytics engine 210 includes a battery life predictor 212 configured to predict a remaining life at a given point of time for batteries in the fleet based on the fleet status information compiled by the fleet status compiler 202. As used herein, the remaining life of a battery refers to an amount of time from the given point of time to when the battery is depleted. In some embodiments, the battery life predictor 212 uses a linear fit method to estimate the remaining life. In some embodiments, the battery life predictor 212 uses a non-linear fit to estimate the remaining life.

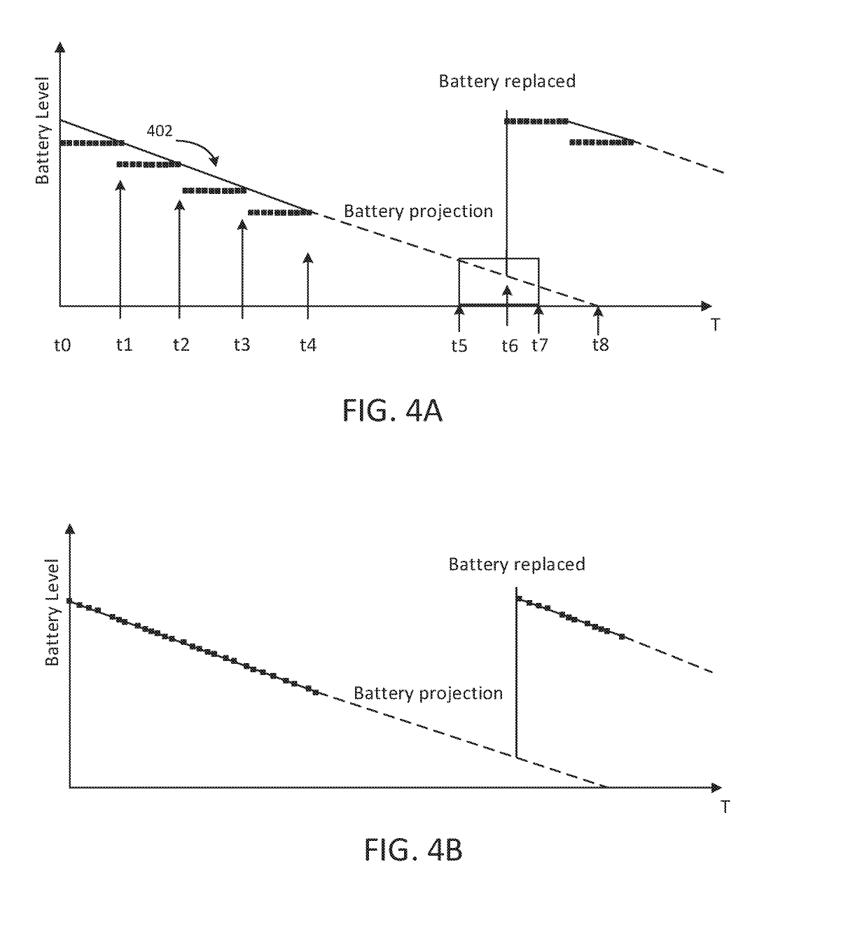

FIG. 4A illustrates fitting battery level readings with a fidelity in ⅛ increments to a battery level decay line, in accordance with an exemplary embodiment. The linear fit method assumes that the battery level decays linearly with time as line 402 shows. At an initial point of time, t0, the battery level reading is 7. At a first point of time, t1, the battery level reading drops to 6. Because the battery level fidelity is in ⅛ increments, during the time period from t0 to t1, any battery level reading is rounded to 7. In other words, if a battery level reading is received at a point of time between t0 and t1, the battery level reading is 7. By the same token, the battery level readings are 6 between t1 and t2, 5 between t2 and t3, 4 between t3 and t4, and so on.

The battery life predictor 212 uses the battery level readings to determine the slope of line 402, which represents the rate of battery level decay. According to line 402, the battery level decays to 0 (i.e., dead) at a point of time t8. The remaining battery life at a given point of time (e.g., t4) is the amount of time between the given point of time (e.g., t4) and t8. An alarm can be generated at a point of time t7, which is, for example, two hours prior to the battery being dead. Thus, if the battery is not replaced before t7, an alarm would be generated at t7 to notify caregivers to change the battery. A change window can be set, which is the desirable period of time to change the battery. If the battery is changed before the change window, considerable battery hours are wasted. If the battery is changed after the change window, an alarm is triggered. In FIG. 4A, for example, the change window is set to be six hours prior to the battery being dead to two hours prior to the battery being dead, i.e., a period of time from t5 to t7. If the battery is changed at a point of time, t6, within the change window, the battery level reading would jump to 7 upon the change. The time period from t6 to t8 is called the wasted battery hours, which is the remaining battery life at t6.

The linear fit method discussed above can be applied to any level of fidelity. For example, FIG. 4B shows fitting battery level readings with a fidelity in 1/100 increments to a battery level decay line in accordance with an exemplary embodiment. The method works the same as discussed above except that the battery level readings are in 1/100 increments.

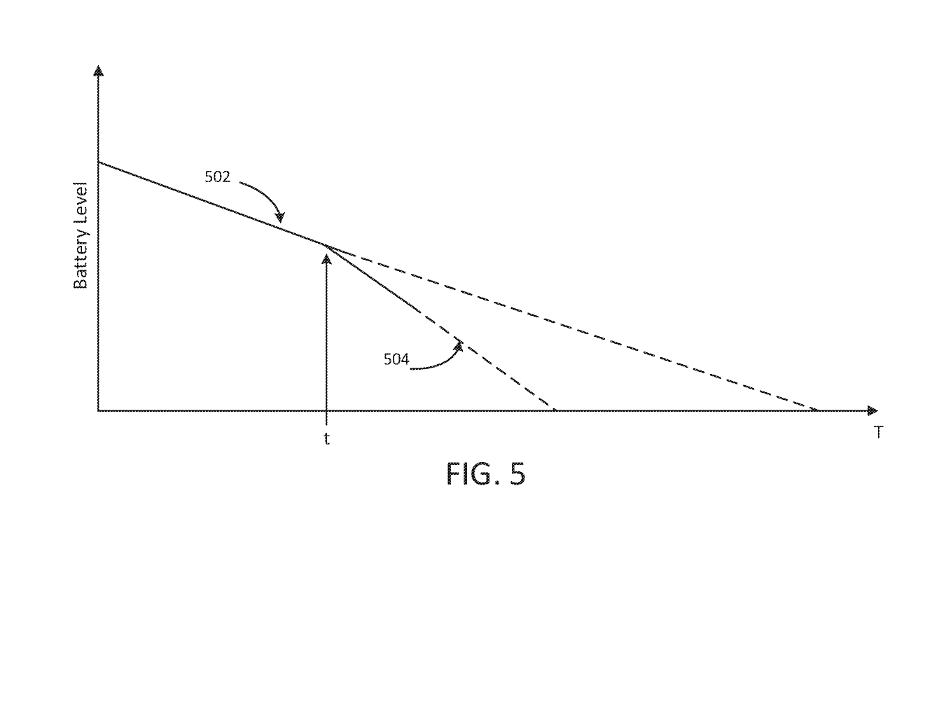

The slope of the battery level decay line, which represents the battery decay rate, may be different for different brands/models/operations of transmitters in which the batteries are used as well as different battery types, brands, etc. For example, different brands/models of transmitters may have different nominal power usage. In addition, the rate of battery power consumption may change when the operation of the transmitter changes. As illustrated in FIG. 5, the transmitter initially monitors ECG of a patient. The battery level decays along line 502 with a first slope. At a point of time, t, SpO2 monitoring (or some other associated monitoring parameter/device) is connected or turned on at the transmitter, therefore the power of the battery is consumed at a faster rate. Starting from t, the battery level decays along line 504 with a second slope. The second slope is steeper than the first slope. In some embodiments, the battery life predictor 212 detects the change of decay rate based on the battery level readings received from the transmitters, which reflect a faster decay rate. In some embodiments, the battery life predictor 212 detects the change based on SpO2-related state changes (e.g., alerts, connected, disconnected) stored in the log file maintained by the telemetry server 130. For example, every time when the SpO2 monitoring is being turned on or off, a SpO2-related alert or state change is recorded in the log file. In some embodiments, the battery life predictor 212 detects the change based on SpO2 readings. For example, when SpO2 monitoring starts, SpO2 readings are generated, which can be identified by the gateway 150. The gateway 150 may then inform the battery life predictor 212 that the SpO2 monitoring is on.

Besides the linear fit method, the battery life predictor 212 may use other methods to predict the remaining battery life such as other data curve fitting or algorithm learning technologies. In some embodiments, the battery life predictor 212 fits the battery level readings to a power decay curve provided by a battery manufacturer to make the prediction. In some embodiments, the battery life predictor 212 fits the battery level readings to a curve learned from historical battery life performance.

It should be understood that the above discussed methods are not limited to disposable batteries, but can be applied to rechargeable batteries with appropriate modifications. For rechargeable batteries, the battery life predictor 212 may obtain, record, and monitor a cycling profile history, fit battery level readings to the decay curve corresponding to the number of charges recorded, and predict remaining life for the current cycling accordingly. Similar battery life prediction approaches mentioned previously for non-rechargeable batteries may also be utilized. The battery management system may also track and alert to when a defined maximum charging cycle has been reached.

Referring back to FIG. 2, the analytics engine 210 includes a fleet report generator 214 configured to generate a report that associates the batteries in the fleet to statuses of corresponding patients and lists actions to be performed on the batteries. In some embodiments, a report is generated automatically when a shift takes place. In some embodiments, a report is generated on demand of a user. In other embodiments, a report, message, alert notification, etc. may be generated in a non-batch mode when the system identifies a status change and impending battery depletion such as when a planned event (e.g., x-ray, dialysis, rehabilitation) occurs or is scheduled.

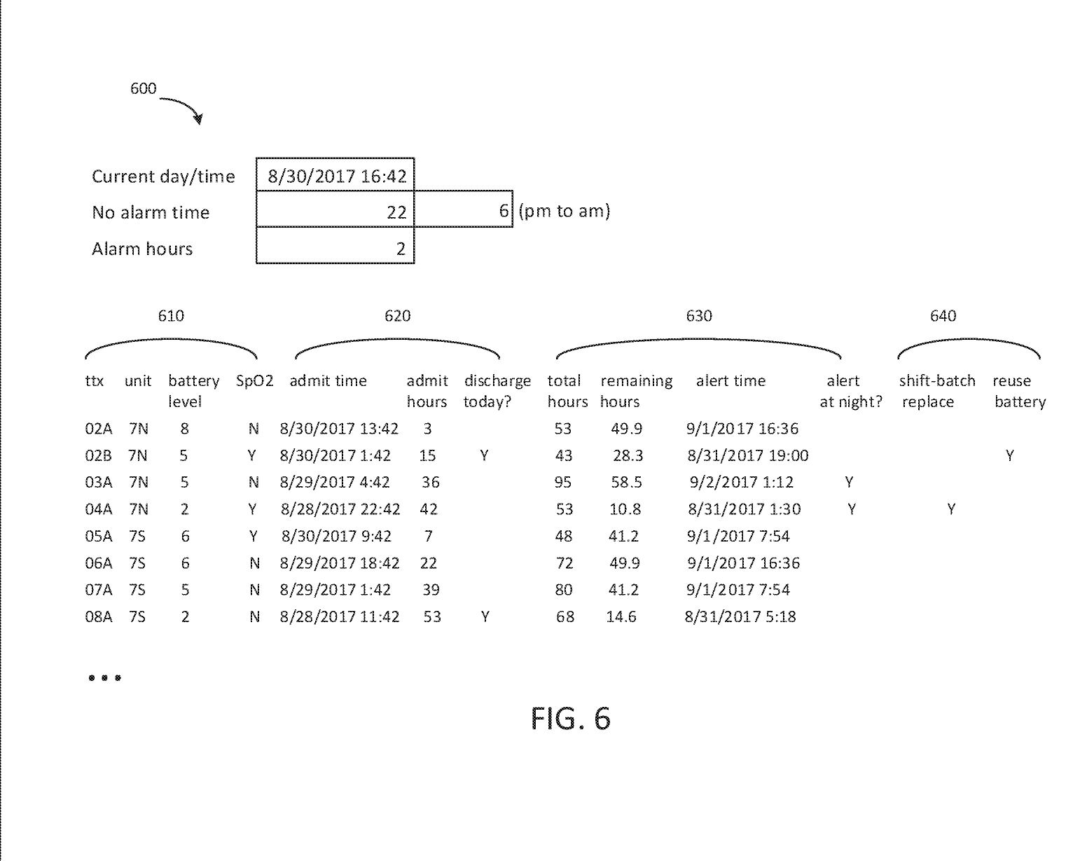

FIG. 6 shows an exemplary fleet report 600 generated by the fleet report generator 214. It should be understood that the report 600 is shown for illustration not for limitation, a report can include fewer, more, or different items than what the report 600 includes and can be of a different layout/configuration. As shown in FIG. 6, in some embodiments, the head of the report 600 includes an item “Current day/time,” which represents the day/time of when the report 600 is generated (e.g., 8/30/2017, 16:42). An item “No alarm time” represents a period of time when alarms relating to batteries are not desired. For example, alarms are not desired from 10:00 PM to 6:00 AM so as not to disturb the patient during sleeping or lights out periods. A user can change the setting of “No alarm time” through the user interface 220. An item “Alarm hours” represents the typical amount of time between when an alarm is generated and when the battery is depleted. This amount of time is often a specification provided by the transmitter vendor and typically varies from manufacturer to manufacturer. As discussed above with reference to FIG. 4A, an alarm would be generated two hours before the battery being dead, thus the “Alarm hours” is 2 (i.e., between t7 and t8). In some embodiments, a user can modify the “Alarm hours” through the user interface 220.

The body of the reporter 600 includes four sections, which are battery information section 610, patient information section 620, battery projection information section 630, and action information section 640. The battery information section 610 includes an item “ttx” representing the ID of the transmitter on which the battery is used, an item “unit” representing the ID of the unit where the transmitter is located, an item “battery level” representing the current reading of battery level, and an item “SpO2” representing whether SpO2 monitoring is on. As discussed above, the transmitter ID, unit ID, and battery level reading can be obtained from the log file maintained by the telemetry server 130, transmitted directly to the battery management system 200 by the transmitters, or like approaches. Whether SpO2 is on can be determined based on battery level decay rate, SpO2-related alert, and/or SpO2 readings, as discussed above.

The patient information section 620 includes an item “admit time” representing when the patient wearing the transmitter was admitted, an item “admit hours” representing how long the patient has been admitted, an item “discharge today?” representing whether the patient will be discharged on the present day. The patient information may be obtained from at least one of the HIS 162, CIS 164, or EMR/EHR 166. In some embodiments, a transmitter is associated with a particular patient through a registration process.

The exemplary battery projection information section 630 includes an item “total hours” representing the total life of the battery, an item “remaining hours” representing the remaining life of the battery, an item “alert time” representing when a battery alert would likely be triggered, and an item “alert at night?” representing whether the predicted alert would happen at night. As discussed above, the battery life predictor 212 can predict the battery life by fitting available battery level readings to a decay model (e.g., a linear decay line, nonlinear decay line, power decay curve provided by a manufacturer, historical performance curve). The total battery life and the remaining battery life can be estimated using the decay model. In some embodiments, the “remaining hours” item indicates the remaining life of the battery at the time of the report being generated. In some embodiments, the “remaining hours” item indicates the remaining life of the battery at the time of scheduled batch replacement. In some embodiments, the “remaining hours” item indicates the remaining life of the battery at the time when a planned event (e.g., x-ray, dialysis, rehabilitation) occurs or is scheduled. In some embodiments, the “remaining hours” item indicates the remaining life of the battery at the time of the associated patient being discharged. In some embodiments, the “remaining hours” item can include any combination of the above.

The “alert time” can be obtained using the “Current day/time,” the “Alarm hours,” and the “remaining hours.” For example, for the first transmitter “02A,” the battery has 49.9 hours remaining. The “Alarm hours” is set as “2,” which means that an alarm is triggered two hours prior to the battery being dead. Therefore, an alarm will be triggered 47.9 hours (i.e., 49.9−2) from the current time (i.e., 8/30/2017 16:42). Therefore, the predicted alarm time is 9/1/2017 16:36. Similar calculation is done for each transmitter.

Whether the predicted alarm would take place at night is determined by comparing the “alert time” to the “No alarm time.” In particular, if the predicted “alarm time” is within the range of the “No alarm time,” the predicted alarm is determined to take place at night. For example, for the transmitter “02A,” the predicted alert time is 16:36, not within the “No alarm time” which is 10 PM to 6 AM, and therefore is not at night. For the transmitter “03A,” the predicted “alarm time” is 1:12, which is within the “No alarm time,” and therefore is determined to be at night. The item “alert at night?” is thus marked as “Y” (i.e., yes).

In some embodiments, the battery projection information section 630 includes an item “alert during planned event” (not shown in the present exemplary figure) representing whether the predicted alert would happen during a planned event, which can be determined by comparing the “alert time” to the time of the planned event.

The action information section 640 includes an item “shift-batch replace” representing whether to replace the battery in a shift-batch replacement event and an item “reuse battery” representing whether to reuse the battery when the associated patient is discharged. In some embodiments, a technician or medical staff replaces a batch of batteries at the time of shift. Batteries can be replaced before an alarm is triggered at night so that disruption to patients can be avoided and alarm fatigue on caregivers can be reduced. For example, if a batched replacement is scheduled to start at 8:30 PM of the present day, batteries that need to be changed are identified at the action information section 640 based on the “alert time” item and the “alert at night” item. If another batched replacement is scheduled at 6:30 AM, batteries that need to be changed can be identified based on the “alert time” item and the “alert during planned event” item. In some embodiments, when a patient is discharged, the battery in the transmitter associated with the patient may still have a meaning remaining life and can be used for next patient. Batteries that can be reused are identified at the “reuse battery” item based on the “remaining hours” item and the “discharge today” item. It should be understood, the exemplary battery management system 200 or battery change manager 216 can make determinations throughout the day, resulting in batched battery replacement recommendations (i.e., multiple transmitters/transceivers) or a single transmitter/transceiver battery replacement recommendation. These recommendations are then in turn communicated to the designated recipient or recipients through various forms including but not limited to printed reports, e-mailed messages and reports, and alerts via a notification system 170.

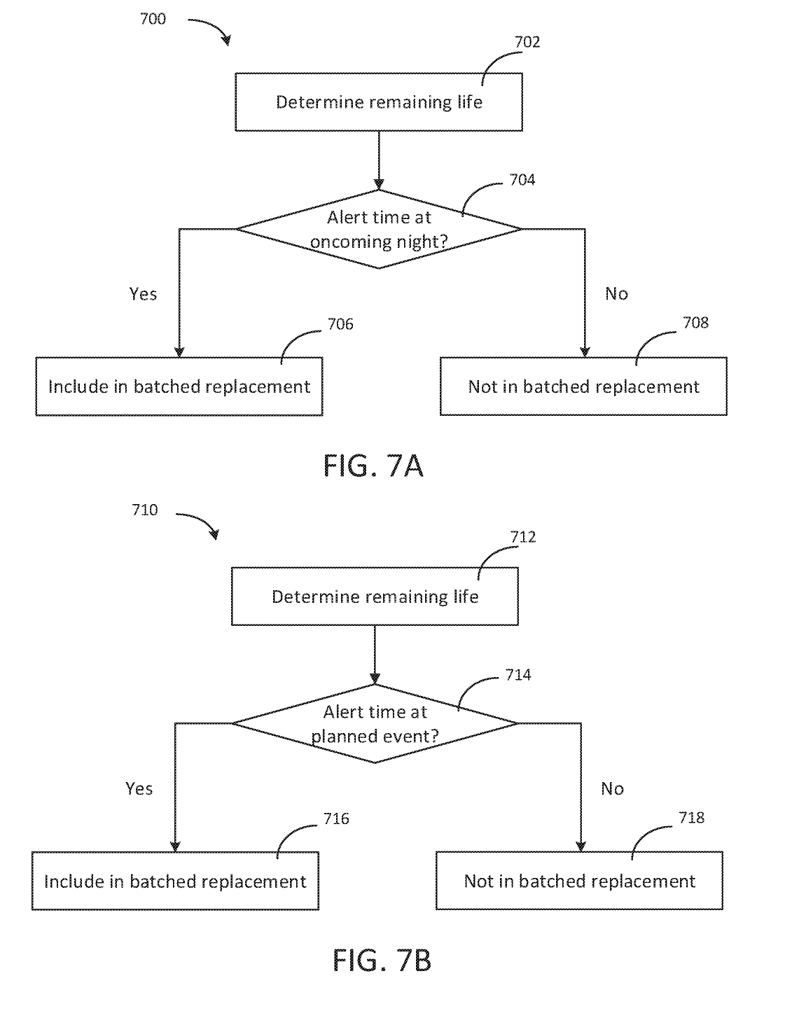

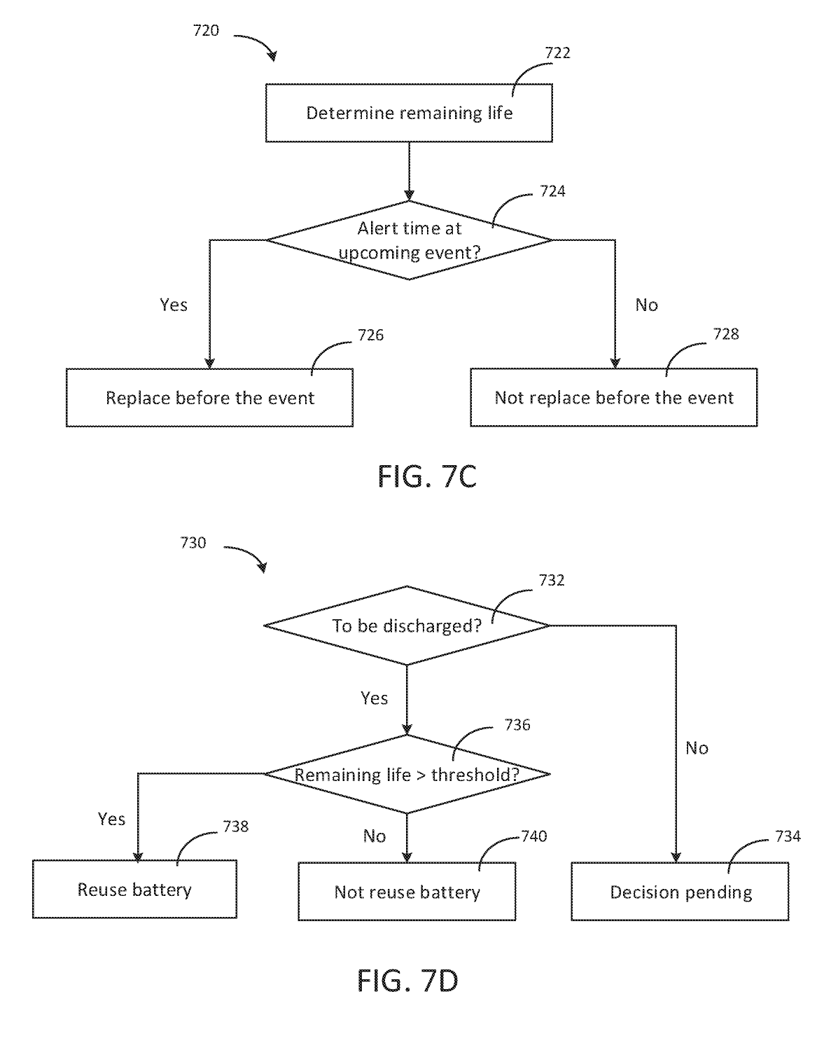

FIG. 7A shows a flow chart 700 for determining whether to replace a battery in a batched replacement event before an alarm is generated, in accordance with an exemplary embodiment. At an operation 702, the remaining life of the battery is determined by fitting available battery level readings to a decay model (e.g., a decay line, power decay curve provided by a manufacturer, historical performance curve), as discussed above. At an operation 704, it is determined whether a predicted alarm would take place during an oncoming night. The predicted alarm is determined to happen during the oncoming night if the “alert time” time falls within the oncoming “No alarm time.” If the predicted alarm would happen during the oncoming night (i.e., “Yes” at operation 704), the battery is included in the batched replacement at operation 706. If the predicted alarm would not happen during the oncoming night (i.e., “No” at operation 704), the battery is not included in the batched replacement at operation 708.

For example, for transmitters “02A,” “02B,” “05A,” “06A,” and “07A,” predicted alarm time is not at night. Thus, batteries used in these transmitters will not be replaced during the shift-batch replacement. For transmitter “03A,” although the predicted alarm time is at night, it is more than two days from the current day/time. Thus, battery used in “03A” will not be replaced during the shift-batch replacement. For transmitter “04A,” the predicted alarm time is in the oncoming night (i.e., 8/31/2017 1:30) and thus, the battery used in “04A” is included in the shift-batch replacement.

FIG. 7B shows a flow chart 710 for determining whether to replace a battery in a batched replacement event before an alarm is generated, in accordance with another exemplary embodiment. At an operation 712, the remaining life of the battery is determined by fitting available battery level readings to a decay model (e.g., a linear decay line, a nonlinear decay line, power decay curve provided by a manufacturer, historical performance curve), as discussed above. At an operation 714, it is determined whether a predicted alarm would take place during a planned event for the associated patient. If the predicted alarm would happen during the planned event (i.e., “Yes” at operation 714), the battery is included in the batched replacement at operation 716. If the predicted alarm would not happen during the planned event (i.e., “No” at operation 714), the battery is not included in the batched replacement at operation 718.

FIG. 7C shows a flow chart 720 for determining whether to replace a battery in a single replacement event before an alarm is generated, in accordance with an exemplary embodiment. Medical staff may order or perform an event for a patient between two batched replacements, e.g., moving the patient to another department, ambulating the patient, x-ray, dialysis, rehabilitation, etc., which event may not be caught and considered in the process 710. The process 720 can be performed dynamically upon the medical staff planning/scheduling/performing the event. At an operation 722, the remaining life of the battery is determined by fitting available battery level readings to a decay model (e.g., a linear decay line, a nonlinear decay line, power decay curve provided by a manufacturer, historical performance curve), as discussed above. At an operation 724, it is determined whether a predicted alarm would take place during an upcoming event for the associated patient. If the predicted alarm would happen during the upcoming event (i.e., “Yes” at operation 724), the battery is replaced before the event at operation 726. If the predicted alarm would not happen during the upcoming event (i.e., “No” at operation 724), the battery is not replaced before the event at operation 728.

FIG. 7D shows a flow chart 730 for determining whether to reuse a battery when a patient associated with the battery is discharged. At an operation 732, it is determined whether the patient is to be discharged, for example, today (i.e., whether the “discharge today” item is marked as “Y”). If the patient is not to be discharged (i.e., “No” at operation 732), the decision of whether to reuse the associated battery will not be made for now but later (operation 734). If the patient is to be discharged (i.e., “Yes” at operation 732), it is determined whether the remaining life the battery at the time of the patient being discharged is larger than a predefined threshold, at an operation 736. The threshold may be the percentage (e.g., 40%) of the total battery life, or an amount of time (e.g., 24 hours), or any other appropriate metric. If the remaining life is larger than the threshold (i.e., “Yes” at operation 736), the battery will be reused when the patient is discharged (operation 738). If the remaining life is not larger than the threshold (i.e., “No” at operation 736), the battery will not be reused when the patient is discharged (operation 740).

For example, patients associated with transmitters “02 W” and “08A” will be discharged by the end of the day (e.g., 5:00 pm). For transmitter “03A,” the remaining life is 28.3 hours, larger than a predefined threshold of 24 hours. Thus, battery used in “03A” will be reused when the patient is discharged. For transmitter “08A,” the remaining life is 14.6 hours, not larger than the threshold, and thus the battery used in “08A” will not be reused when the patient is discharged.

Referring back to FIG. 2, the analytics engine 210 includes a battery change manager 216 configured to manage the battery replacement. Both batched replacement and single battery replacement can be managed, as discussed above. In some embodiments, the battery change manager 216 sends the battery change notification to a monitor technician through the alert/alarm notification management system 170 (e.g., ASCOM alarm notification system). In some embodiments, the battery change manager 216 commands the center operational dashboard with conditional alarm notifications.

In some embodiments, the battery change manager 216 schedules a batched-replacement event. For example, the battery change manager 216 may perform the method 700 and 710 as discussed above with reference to FIGS. 7A-7B. As another example, a batched replacement event can be scheduled when a certain number (e.g., 10, 20, etc.) of batteries have entered the battery change window (e.g., the time period from t5 to t7 as shown in FIG. 4A). The batched replacement may be created in different groupings based on the intended audience such as but not limited to at the nurse, partial unit, unit, floor, or hospital level. In some embodiments, a single notification regarding a batched-replacement event is sent out.

In some embodiments, the battery change manger 216 schedules a single replacement event, for example, as discussed above with reference to FIG. 7C. In some embodiments, the battery change manager 216 determines whether to replace or reuse a battery when the associated patient is discharged, as discussed above with reference to FIG. 7D.

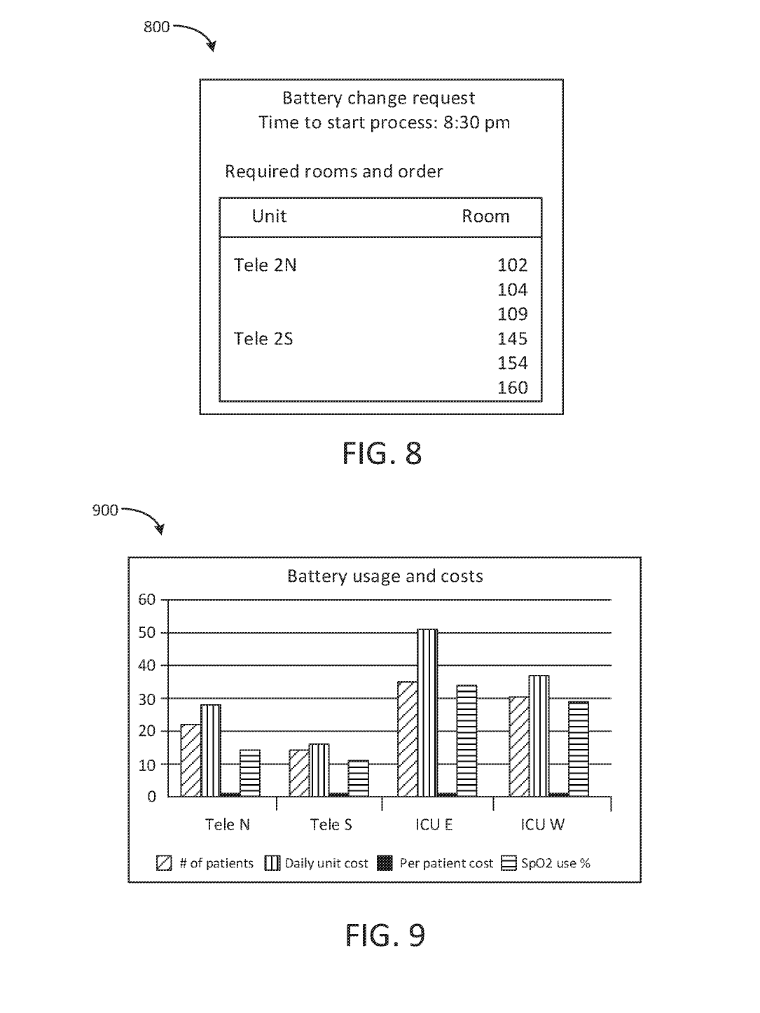

In further embodiments, the battery change manager 216 plans an optimized route for the batched replacement that takes the least time, shortest walk distance, etc. FIG. 8 shows an exemplary printed order 800 with a travel path. Printed order 800 shows first changing the batteries in Room 102 of Unit 2N, then Room 104, and Room 109, then changing the batteries in Room 145 of Unit 2S, Room 154, Room 160, and so on. When planning the route, the battery change manager 216 refers to a hospital plan/layout. In some embodiments, the hospital plan/layout is stored in the storage 230 of the battery management system 200. In some embodiments, the battery change manager 216 accesses the hospital plan/layout stored elsewhere.

In some embodiments, the battery change manager 216 determines when and by whom to replace the batteries by taking into account multiple factors. For example, the battery change manager 216 can put time constraints on a battery change. If a patient will be away from the room for scheduled physical therapy or order (e.g., dialysis, X-ray), the battery change manage 216 determines if the battery may deplete completely or to a warning/alerting level during the time and plans a battery change prior to the planned activity, procedure, etc. The location of the patient can be obtained from, for example, the location service system 168. The location information may be included in battery change reporting and alerting, used to identify situations where the patient is not in their room when a battery change may take place, and the like. As another example, there are “rush hours” when elevators to some particular units are busy. The battery change manager 216 can plan changing batteries for those units before or after the rush hours of elevators.

In some embodiments, the battery change manager 216 determines by whom to replace the battery based on costs and availability of staff. The care team may include various members such as patient care technicians, monitor technicians who are available for battery replacement. In some embodiments, the battery change manager 216 chooses a team member with the lowest cost to replace the batteries. In some embodiments, the battery change manager 216 chooses a team member who is closest to the units to replace the batteries.

In some embodiments, the battery change manager 216 confirms that a battery has been replaced so that a replace/alarm notification can be removed. For example, if the battery level reading of a transmitter jumps to a high value, the battery change manager 216 determines that the battery has been replaced for the transmitter and any replace/alarm notification relating to batteries can be removed for that transmitter.

Referring back to FIG. 2, the analytics engine 210 further includes an operation manage 216 configured to make operational decisions and set operational policies for the fleet of batteries. In some embodiments, the operation manager 216 conducts statistical analyses on operational metrics of the fleet and performs optimizations to enhance the operational effectiveness of the monitoring system based on the statistical analyses. The exemplary operational metrics include but are not limited to: battery acquisition cost (e.g., for a year of operation), battery change cost (e.g., compensation to monitor technicians for performing the replacement), numbers and/or percentage of batteries that are in use in the fleet, numbers and/or percentage of batteries in stock; average and/or total wasted battery life (i.e., remaining battery life in a battery being replaced and not reused), numbers and/or percentage of batteries reused after associated patients were discharged, counts of alarms, counts and/or percentage of changes before alarms being triggered, counts and/or percentage or changes during restricted hours (e.g., late night), counts and/or percentage of battery replaced before restricted hours, suggested battery change compliance and timing, etc. The operational statistics can be stored in the storage 230 of the battery management system 200 or elsewhere and updated daily, weekly, monthly, annually, and so on.

In some embodiments, the operational statistics can be presented to a user through the user interface 220 as charts. FIG. 9 shows an exemplary chart 900 which illustrates statistics on battery usage and costs breakdown by unit. For each unit, the chart 900 shows the number of patients, daily unit cost, per patient cost, and percentage of SpO2 usage.

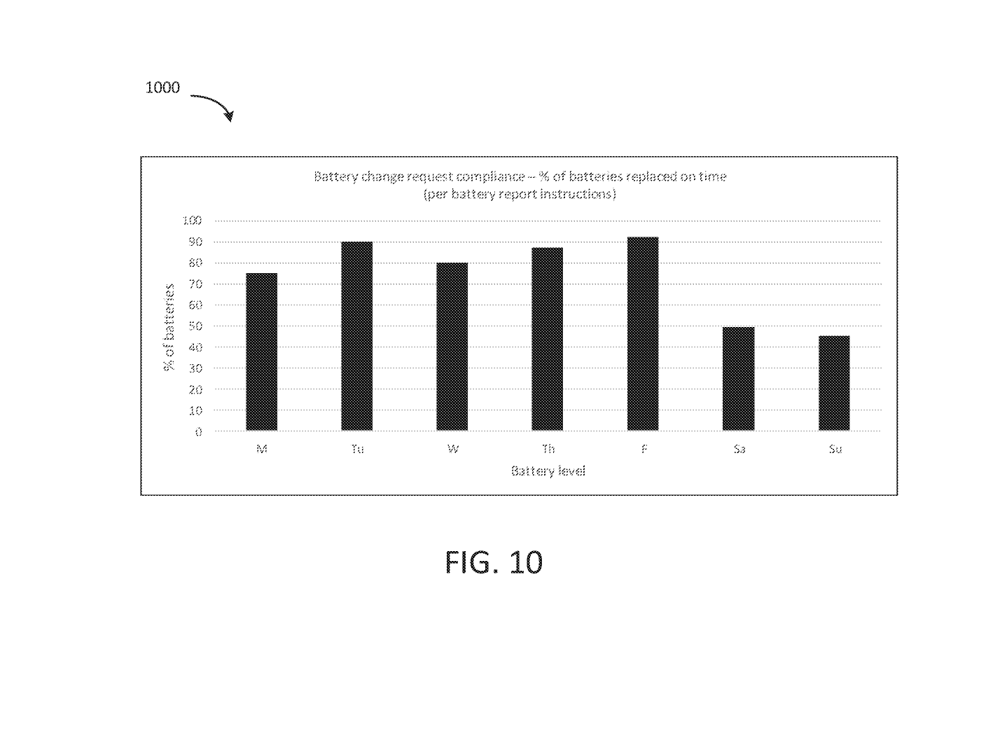

FIG. 10 shows an exemplary chart 1000 which illustrates statistics on battery change request compliance, i.e., the percentage of batteries replaced on time per battery report instructions. It shows that better compliance is achieved during weekdays (i.e., Monday through Friday) than weekends (i.e., Saturday and Sunday) due to, for example, use of temporary nursing staff who may not have the same level of procedural knowledge or familiarity or those who may not have proper training.

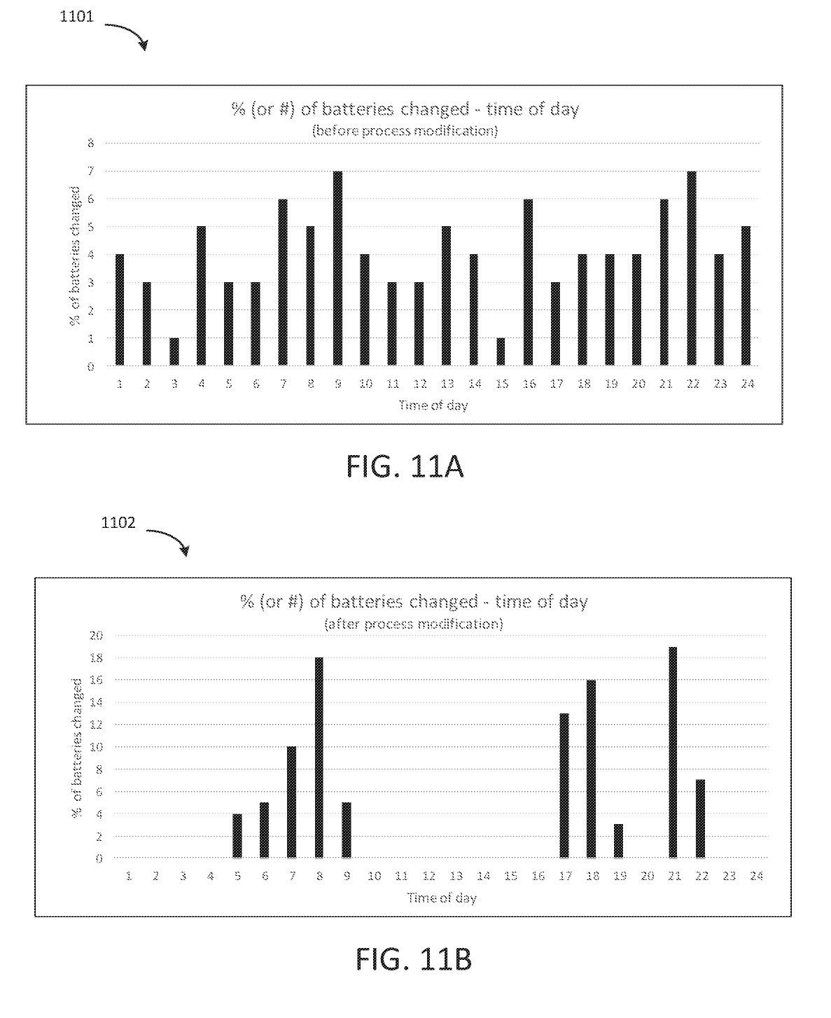

FIGS. 11A and 11B show exemplary charts 1101 and 1102 which illustrate statistics on battery replacement breakdown by the time of day before and after the replacement process was modified by the battery management system. Chart 1101 shows that batteries are changed in a relatively evenly fashion throughout the day before the process 700 is implemented. Chart 1102 shows that very few batteries (in terms of numbers/percentage) are changed during the night time (i.e., 10:00 pm-6:00 am) because, for example, the process 700 (shown in FIG. 7A) is implemented. Thus, disruption to patients can be reduced.

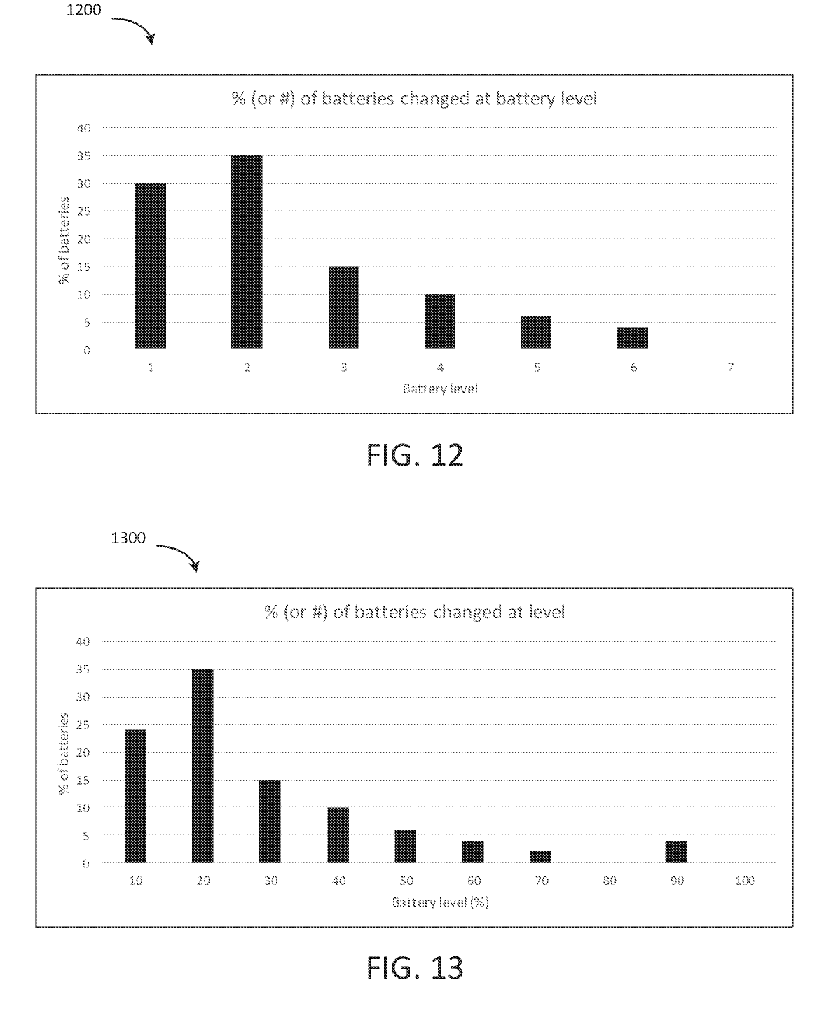

FIG. 12 shows an exemplary chart 1200 which illustrates statistics on batteries changed at various remaining battery life in terms of battery level. FIG. 13 shows an exemplary chart 1300 which illustrates statistics on batteries changed at various remaining battery life in terms of percentage of total battery life. These charts can be used to evaluate wasted battery hours.

It should be understood that FIGS. 9-12 are shown for illustration not for limitation, any other chart can show, for example, statistics at the hospital level of various metrics.

In some embodiments, the operation manager 216 uses the acquired information, statistics, associated information sources (e.g., patient satisfaction ratings), etc. along with optimization techniques to guide or make operational decisions. For example, the operation manager 216 can determine the budget based on historical costs of ownership, patient levels, patient classifications (i.e., percentage of SpO2 usage, length of stay), and so on. As another example, the operation manager 216 can decide when to reorder batteries based on numbers/percentage of batteries in use and in stock. For example, the operation manager 216 may decide to reorder batteries when batteries in stock will be used up in, for example, a week, two weeks, a month, etc.

In some embodiments, the operation manager 216 sets operational policies and/or strategies to meet the hospital's or clinic's goals. Hospitals and clinics have multiple goals to achieve with different priorities. Exemplary goals include but are not limited to: achieving desired Hospital Consumer Assessment of Healthcare Providers and Systems (HCAHPS) scores, reducing costs, increasing process efficiency (e.g., device battery management) so clinician time can be more effectively used treating patients, reducing alarm fatigue on medical professionals and staff, and so on. Some clinics may set HCAHPS score as the highest priority. Some hospitals/clinics are willing to trade some patient satisfaction for cost savings. Other hospitals/clinics desire to minimize technical alarms at the price of higher battery and operating costs. The operation manager 216 may have different policies in place according to the priorities of different hospitals/clinics. For example, as discussed above, a restricted time period (e.g., late night) can be set during which no alarm and no battery change should happen. If an alarm is predicted to be generated during the restricted time period, the battery would be changed before the restricted time period. For example, the restricted time period can be set longer (e.g., from 10 PM to 6 PM) for clinics that have higher expectation on the HCAHPS score, but shorter (e.g., from 11 PM to 5 AM) for clinics that have lower expectation on the HCAHPS score.

As another example, if reducing technical alarms is of high priority, the operation manager 216 may set a policy of replacing batteries that have not yet reached the 2 hour alarms in a batched replacement. If reducing technical alarms is not as high priority as saving battery costs, the policy may allow 2 hour alarms.

As another example, if the clinic is endeavoring to reduce environmental waste, the operation manager 216 may set a policy of reusing batteries. For example, if the estimated remaining battery life is longer than a certain amount of time (e.g., 20 hours, 30 hours, 40 hours, etc.) when the associated patient is discharged, the batteries will be indicated for reuse with the next patient. Whether to reuse the batteries can be presented on the discharge order of the patient. The operational policies can be stored in the storage 230 of the battery management system 200 or elsewhere.

For each exemplary scenario, the input parameters, constraints, and desired objective(s) are envisioned to be modeled and optimized for the ideal recommended solution. In a non-limited example as in the case of determining whether a battery or set of batteries should be kept as opposed to discarded, the remaining cutoff hours may be determined by examining a number of factors (e.g., battery costs, technician costs—time prepare, change, transit, etc., disposal costs, remaining battery life) and using an optimization algorithm to optimize the cutoff hours based on the factors.

As another example, the battery manufacturer/model used by the hospital/clinic may be determined by examining factors of batteries from various manufacturers and of various models, such as the battery cost, decay time with and without SpO2, shift changes, and so on and using an optimization algorithm.

As another example that is discussed above, the routing for battery change request may be determined by considering multiple factors such as battery status, room location, time of day, elevator usage, list of nurse, unit, level, etc. The battery change time periods can be evaluated and determined based on factors such as efficiency, staying away from nights, shift changes, etc. and using an optimization algorithm.

It should be understood the optimization of such these scenarios (and others) can include fewer, more, or different factors than what has been described herein. The outcome of the optimization can be printed out, viewed via a user interface, sent to medical staff and administrators as email, messages, or the like.

Oftentimes, it might be unclear which policy/strategy would be able to achieve more desirable performance of the fleet due to complex situations. A test can run, for example, on two or more difference units at the same period time or on the same unit at different periods of time, to evaluate the impact of different policies. For example, unit A runs a first policy/strategy for a month, unit B runs a second policy/strategy during the same period of time. Operational metrics (e.g., battery costs, staffing costs, patient disruptions and influence on HCAHPS scores, etc.) of unit A are then compared to operational metrics of unit B. Or, unit A runs a first policy/strategy for a first month, and a second policy/strategy for a second month. Operational metrics of the first month are compared to operational metrics of the second month.

As another example, the operation manager 216 helps decide whether to switch to another brand or model of batteries to optimize total cost of ownership (TCO). A test can run for the old brand/model and a proposed new brand/model of batteries for a period of time. TCO of using the new brand/model is compared to TCO of using the old brand/model. If the TCO for the new brand/model is lower, the clinic may decide to switch to the new brand/model.

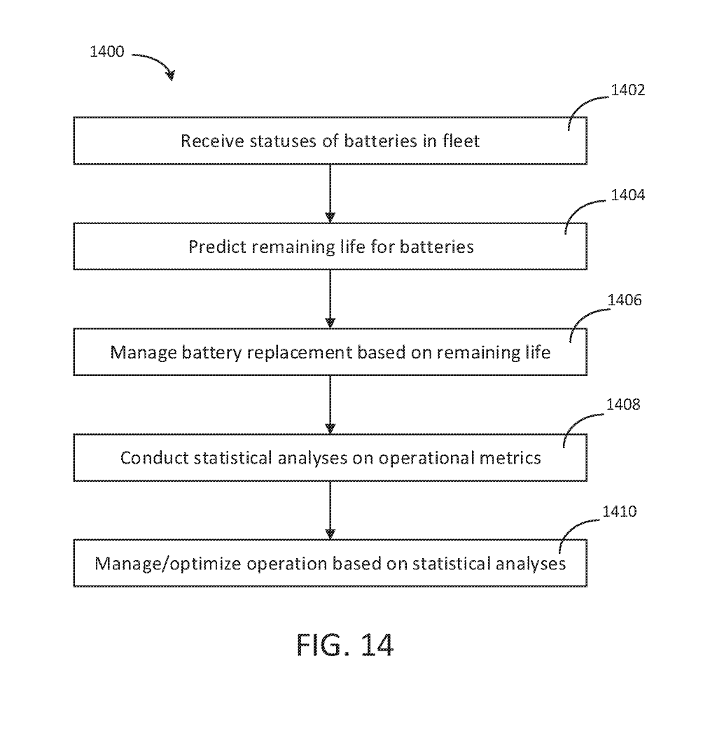

Referring to FIG. 14, a flow chart 1400 of a method for managing a fleet of batteries used in a clinical telemetry patient monitoring system is shown, in accordance with an exemplary embodiment. The method can be performed by the battery management system 200 shown in FIG. 2.

At an operation 1402, statuses of batteries in the fleet is received from transmitters on which the batteries are used. Each of the transmitters is associated with a corresponding patient for monitoring health of the patient. The statuses of batteries include battery levels measured at various points of time for each of the batteries (i.e., battery level readings).

At an operation 1404, a remaining life at a given point of time is predicted for each of the batteries based on the received statuses. In some embodiments, the measured battery levels are fit to a decay model (e.g., linear model, non-liner model, learned decay curves, manufacturer provided decay curves) and the remaining life at the given point of time is calculated using the decay model.

At an operation 1406, battery replacement is managed based on the remaining life. In some embodiments, an alarm time of when an alarm for battery replacement would be generated is predicted for a particular battery based on the remaining life of the particular battery. It is determined whether the alarm time falls within a predefined restriction time period. If the alarm time falls within the predefined restriction time period, a replacement is scheduled for the particular battery at a point of time before the predefined restriction time period. In some embodiments, a route for a batched replacement of batteries is planned. In some embodiments, the remaining life of the battery at the point of time when a patient associated with the transmitter in which the battery is to be charged is compared with a predefined threshold. If the remaining life is larger than the threshold, the battery would not be replaced when the patient is discharged but be used for the next patient. If the remaining life is not larger than the threshold, the battery would be replaced upon discharge of the patient.

At an operation 1408, statistical analyses and optimization are conducted on operational metrics of the fleet of batteries. In some embodiments, the operation metrics comprises at least some of battery acquisition cost, staff cost, total cost of ownership, numbers/percentage of batteries in use, numbers and/or percentage of batteries in stock, average/total wasted battery life, numbers/percentage of batteries reused after associated patients were discharged, counts of alarms, counts/percentage of changes before alarms being triggered, counts/percentage or changes during restricted hours, counts/percentage of battery replaced before restricted hours.

At an operation 1410, operation of the fleet of batteries is managed based on the statistical analyses. In some embodiments, a battery order time is predicted based on the number/percentage of batteries in use and in stock. In some embodiments, policies are set to optimize at least some of the operation metrics. For example, a first set of statistical analyses are performed on the operation metrics for the fleet of batteries operated in accordance with a first policy/strategy. A second set of statistical analyses are performed on the operation metrics for the fleet of batteries operated in accordance with a second policy/strategy. The first set of statistical analyses are compared to the second set of statistical analyses and one policy/strategy is implemented based on the comparison.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

What is claimed is:

1. A method for managing a fleet of batteries used in a clinical telemetry patient monitoring system, the method comprising:
receiving statuses of batteries in the fleet from transmitters and/or transceivers in which the batteries are used, wherein each transmitter or transceiver is associated with a corresponding patient for monitoring physiological conditions of the patient;
predicting a remaining life at a given point of time for each of the batteries based on the received statuses; and
managing battery replacement based on the remaining life.

2. The method of claim 1, wherein the statuses of batteries include battery levels measured at various points of time for each of the batteries.

3. The method of claim 2, wherein predicting the remaining life comprises:
fitting the battery levels to a model; and
using the model to calculate the remaining life at the given point of time.

4. The method of claim 1, wherein managing battery replacement based on the remaining life comprises:
predicting an alarm time of when an alarm for battery replacement is generated for a particular battery based on the remaining life of the particular battery;
determining whether the alarm time falls within a predefined restriction time period or an event; and
scheduling to replace the particular battery at a point of time before the predefined restriction time period or the event in response to determining that the alarm time falls within the predefined restriction time period or the event.

5. The method of claim 1, wherein managing battery replacement based on the remaining life comprises:
comparing the remaining life of a particular battery at a point of time when the corresponding patient is discharged with a predefined threshold; and
reusing the particular battery in response to the remaining life being larger than the predefined threshold.

6. The method of claim 1, wherein managing battery replacement comprises planning a route for a replacement of batteries.

7. The method of claim 1, further comprising:
conducting statistical analyses on operational metrics of the fleet of batteries; and
managing operation of the fleet of batteries based on the statistical analyses.

8. The method of claim 7, wherein the operational metrics comprise at least some of battery acquisition cost, staff cost, total cost of ownership, numbers/percentage of batteries in use, numbers and/or percentage of batteries in stock, average/total wasted battery life, numbers/percentage of batteries reused after associated patients were discharged, battery change compliance and timing, counts of alarms, counts/percentage of changes before alarms being triggered, counts/percentage or changes during restricted hours, counts/percentage of battery replaced before restricted hours.

9. The method of claim 8, wherein managing the operation of a fleet of batteries comprises predicting a battery order time based on the number/percentage of batteries in use and in stock.

10. The method of claim 7, wherein managing the operation of a fleet of batteries comprises setting policies and/or strategies to optimize operations based on at least some of the operational metrics.

11. The method of claim 7, wherein managing the operation of a fleet of batteries comprises:
performing a first set of statistical analyses on the operational metrics for a fleet of batteries operated in accordance with a first policy/strategy;
performing a second set of statistical analyses on the operational metrics for a fleet of batteries operated in accordance with a second policy/strategy;
comparing the first set of statistical analyses to the second set of statistical analyses; and
implementing one of the first policy/strategy and the second policy/strategy based on the comparison.

12. A processing system for managing a fleet of batteries used in a telemetry clinical patient monitoring system, the processing system comprises:
a fleet status compiler configured to compile statuses of batteries in the fleet collected from transmitters and/or transceivers in which the batteries are used, wherein each transmitter or transceiver is associated with a corresponding patient for monitoring physiological conditions of the patient;
and an analytics engine configured to:
predict a remaining life at a given point of time for each of the batteries based on the statuses of batteries; and
manage battery replacement based on the remaining life.

13. The processing system of claim 12, wherein the statuses of batteries include battery levels measured at various points of time for each of the batteries.

14. The processing system of claim 13, wherein predicting the remaining life comprises:
fitting the battery levels to a model; and
using the model to calculate the remaining life at the given point of time.

15. The processing system of claim 12, wherein managing battery replacement based on the remaining life comprises:
predicting an alarm time of when an alarm for battery replacement is generated for a particular battery based on the remaining life of the particular battery;
determining whether the alarm time falls within a predefined restriction time period or an event; and
scheduling to replace the particular battery at a point of time before the predefined restriction time period or the event in response to determining that the alarm time falls within the predefined restriction time period or the event.

16. The processing system of claim 12, wherein managing battery replacement based on the remaining life comprises:
comparing the remaining life of a particular battery at a point of time when the corresponding patient is discharged with a predefined threshold; and
reusing the particular battery in response to the remaining life being larger than the predefined threshold.

17. The processing system of claim 12, wherein the analytics engine is further configured to:
conduct statistical analyses on operational metrics of the fleet of batteries; and
manage operation of the fleet of batteries based on the statistical analyses.

18. The processing system of claim 17, further comprising a user interface configured to present the statistics on at least some of the operational metrics to a user.

19. The processing system of claim 17, wherein managing operation of the fleet of batteries comprises setting policies and/or strategies to optimize operation based on at least some of the operation metrics.

20. A non-transitory computer readable medium comprising instructions which, when executed by a processing system, cause the processing system to perform operations comprising:
receiving statuses of batteries in a fleet from transmitters and/or transceivers in which the batteries are used, wherein each transmitter or transceiver is associated with a corresponding patient for monitoring health of the patient;
predicting a remaining life at a given point of time for each of the batteries based on the received status; and
managing battery replacement based on the remaining life.
